(19) United States
(12) Patent Application Publication
(10) Pub. No.: US 2020/0321097 A1
(43) Pub. Date: Oct. 8, 2020

(54) SYSTEM, DEVICE, AND METHODS FOR PROVIDING DIETARY AND HEALTH INFORMATION

(71) Applicant: Sahith Kudaravalli, Fremont, CA (US)

(72) Inventor: Sahith Kudaravalli, Fremont, CA (US)

(21) Appl. No.: 16/837,330

(22) Filed: Apr. 1, 2020

Related U.S. Application Data
(60) Provisional application No. 62/828,197, filed on Apr. 2, 2019.
(60) Provisional application No. 62/939,977, filed on Nov. 25, 2019.

(51) Int. Cl. G16H 20/60; G16H 10/60; G16H 40/67; G16H 20/30; G16H 50/70; G16H 50/30; A61B 5/022; A61B 5/145; G01G 19/44; G09B 19/00

(52) U.S. Cl. CPC G16H 20/60; G16H 10/60; G16H 40/67; G16H 20/30; G09B 19/0092; G16H 50/30; A61B 5/022; A61B 5/14532; G01G 19/44; G16H 50/70

(57) ABSTRACT

A method for providing dietary health and progress information to a user may include the steps of: receiving input from a user through a client device, in which the input includes a biometric data goal; receiving baseline health information of the user via a health monitoring device, the baseline health information including a first biometric data, the first biometric data communicated from the health monitoring device to the client device; receiving user nutrition and exercise data via the client device; and providing a first modification to the user via the client device, in which the first modification includes a health recommendation to achieve the biometric data goal of the user.

### CROSS REFERENCE TO RELATED APPLICATIONS

This application claims priority to and the benefit of the filing date of U.S. Provisional Application No. 62/828,197, filed on Apr. 02, 2017, entitled “SYSTEM, DEVICE, AND METHODS FOR PROVIDING DIETARY AND HEALTH INFORMATION”, which is hereby incorporated by reference in its entirety. This application also claims priority to and the benefit of the filing date of U.S. Provisional Application No. 62/939,977, filed on Nov. 25, 2019, entitled “SYSTEM, DEVICE, AND METHODS FOR PROVIDING DIETARY AND HEALTH INFORMATION”, which is hereby incorporated by reference in its entirety.

### FIELD OF THE INVENTION

This patent specification relates to the field of healthy eating and providing health information to a user. More specifically, this patent specification relates to systems, devices, and methods for providing freshly ground and mixed, raw form spices and other edible nutritional items as well as systems for providing dietary health information to a user.

### BACKGROUND

While it is known that spices and other edible nutritional items are able to provide positive health benefits, most individuals find it difficult to prepare and utilize one or more freshly ground and mixed, raw form spices and other edible nutritional items. For this reason, they resort to using pre-ground or prepared edible nutritional items which are of limited freshness and flavor. Furthermore, it can be difficult to remember and consistently repeat mixtures of edible nutritional items.

Therefore, a need exists for novel systems, devices, and methods for providing freshly ground and mixed spices as well as dietary health and information to a user. A further need exists for novel systems, devices, and methods for providing dietary health and information which may be utilized by a user to naturally cure health problem(s). Still a further need exists for novel systems, devices, and methods for providing dietary health and information which may be utilized by a user to achieve healthier eating habits and exercise to lose weight and maintain good cholesterol, glucose, blood pressure, and sugar levels. Another need exists for novel systems, devices, and methods for providing dietary health and information to a user which are able to provide freshly ground and mixed, raw form spices and other edible nutritional items having the freshest and most robust flavors. Yet a further need exists for novel systems, devices, and methods for providing dietary health and information to a user which are able to store various mixtures of edible nutritional items and to consistently reproduce and provide the various mixtures of edible nutritional items to users.

### BRIEF SUMMARY OF THE INVENTION

A novel system, device, and methods for providing healthy edible items such as spices and dietary health and information which may be utilized by a user to naturally cure health problem(s) are provided which may be utilized by a user to achieve healthier eating habits and exercise to lose weight and maintain good cholesterol, glucose, blood pressure, and sugar levels.

In some embodiments, a method for providing dietary health and progress information to a user may include the steps of: receiving user data via a client device; receiving baseline health information of user via a client device and a health monitoring device; receiving user nutrition and exercise data via a client device and a dietary metering device; calculating the health progress of the user; providing health progress of the user via a client device; and providing modifications to user, via a client device, the modifications used to meet health goals of the user.

In further embodiments, a method for providing dietary health and progress information to a user may include the steps of: receiving input from a user through a client device, in which the input includes a biometric data goal; receiving baseline health information of the user via a health monitoring device, the baseline health information including a first biometric data, the first biometric data communicated from the health monitoring device to the client device; receiving user nutrition and exercise data via the client device; and providing a first modification to the user via the client device, in which the first modification includes a health recommendation to achieve the biometric data goal of the user.

In some embodiments, a system for providing dietary and health information may include: one or more dietary metering devices which may be configured to dispense metered amounts of edible nutritional items; one or more health monitoring devices which may be configured to provide health biometric information of a user; and one or more client devices which may be in communication with the dietary metering devices and/or health monitoring devices, the client device configured to provide health progress and modifications to a user which may be used to meet health goal of the user.

In further embodiments, a system for providing dietary and health information may include: a client device having a display screen and an input interface for receiving input from a user; and a computing platform having a processor, a memory in communication with the processor. A communication logic may be stored in the memory, executable by the processor and configured to receive input from the user, the input including a biometric data goal, and configured to receive baseline health information of the user via a health monitoring device, the baseline health information including a first biometric data, the first biometric data communicated from the health monitoring device to the client device. An acquisition logic may be stored in the memory, executable by the processor and configured to receive user nutrition and exercise data via the client device. An analytics logic may be stored in the memory, executable by the processor and configured to generate a first modification, in which the first modification includes a health recommendation to achieve the biometric data goal of the user, and in which the first modification is provided to the user via the communication logic.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts an illustrative example of some of the components and computer implemented methods which may be found in a system for providing dietary and health information according to various embodiments described herein.

FIG. 2 illustrates a block diagram showing an example of a server which may be used by the system as described in various embodiments herein.

FIG. 3 shows a block diagram illustrating an example of a client device which may be used by the system as described in various embodiments herein.

FIG. 4 depicts a perspective view of an example of a dietary metering device according to various embodiments described herein.

FIG. 5 illustrates a perspective view of some of the internal components of an example of a dietary metering device according to various embodiments described herein according to various embodiments described herein.

FIG. 6 shows a block diagram of an example of a dietary metering device according to various embodiments described herein according to various embodiments described herein.

FIG. 7 depicts a block diagram of an example of a method for dispensing edible nutritional items from a dietary metering device according to various embodiments described herein.

FIG. 8 illustrates a block diagram of an example of a method for providing dietary health and information according to various embodiments described herein.

FIG. 9 shows a perspective view of some of the internal components of another example of a dietary metering device according to various embodiments described herein according to various embodiments described herein.

FIG. 10 depicts a block diagram of another example of a method for dispensing edible nutritional items from a dietary metering device according to various embodiments described herein.

FIG. 11 shows a block diagram illustrating some applications of a system for providing dietary and health information which may function as software rules engines according to various embodiments described herein.

FIG. 12 illustrates a block diagram of an example of a computer-implemented method for generating one or more modifications according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Although the terms “first”, “second”, etc. are used herein to describe various elements, these elements should not be limited by these terms. These terms are only used to distinguish one element from another element. For example, the first element may be designated as the second element, and the second element may be likewise designated as the first element without departing from the scope of the invention.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

### DEFINITIONS

As used herein, the term “computer” refers to a machine, apparatus, or device that is capable of accepting and performing logic operations from software code. The term “application”, “software”, “software code”, “source code”, “script”, or “computer software” refers to any set of instructions operable to cause a computer to perform an operation. Software code may be operated on by a “rules engine” or processor. Thus, the methods and systems of the present invention may be performed by a computer based on instructions received by computer software.

The term “electronic device” as used herein is a type of computer comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of electronic devices include: personal computers (PCs), workstations, servers, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, wearable computers such as Apple Watch, other smartwatches, Fitbit, other wearable fitness trackers, Google Glasses, and the like.

The term “client device” as used herein is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of client devices include: personal computers (PCs), workstations, servers, laptops, tablet PCs including the iPad, cell phones including iOS phones made by Apple Inc., Android OS phones, Microsoft OS phones, Blackberry phones, Apple iPads, Anota digital pens, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, tablets, digital pens, wearable computers such as Apple Watch, other smartwatches, Fitbit, other wearable fitness trackers, Google Glasses, and the like.

The term “computer readable medium” as used herein refers to any medium that participates in providing instructions to the processor for execution. A computer readable medium may take many forms, including but not limited to, non-volatile media, volatile media, and transmission media. Non-volatile media includes, for example, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications.

As used herein the term “data network” or “network” shall mean an infrastructure capable of connecting two or more computers such as client devices either using wires or wirelessly allowing them to transmit and receive data. Non-limiting examples of data networks may include the internet or wireless networks or (i.e. a “wireless network”) which may include Wifi and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigby network, or a voice-over-IP (VoIP) network.

As used herein, the term “database” shall generally mean a digital collection of data or information. The present invention uses novel methods and processes to store, link, and modify information such digital images and videos and user profile information. For the purposes of the present disclosure, a database may be stored on a remote server and accessed by a client device through the internet (i.e., the database is in the cloud) or alternatively in some embodiments the database may be stored on the client device or remote computer itself (i.e., local storage). A “data store” as used herein may contain or comprise a database (i.e. information and data from a database may be recorded into a medium on a data store).

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

A new system, device, and methods for providing dietary and health information are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

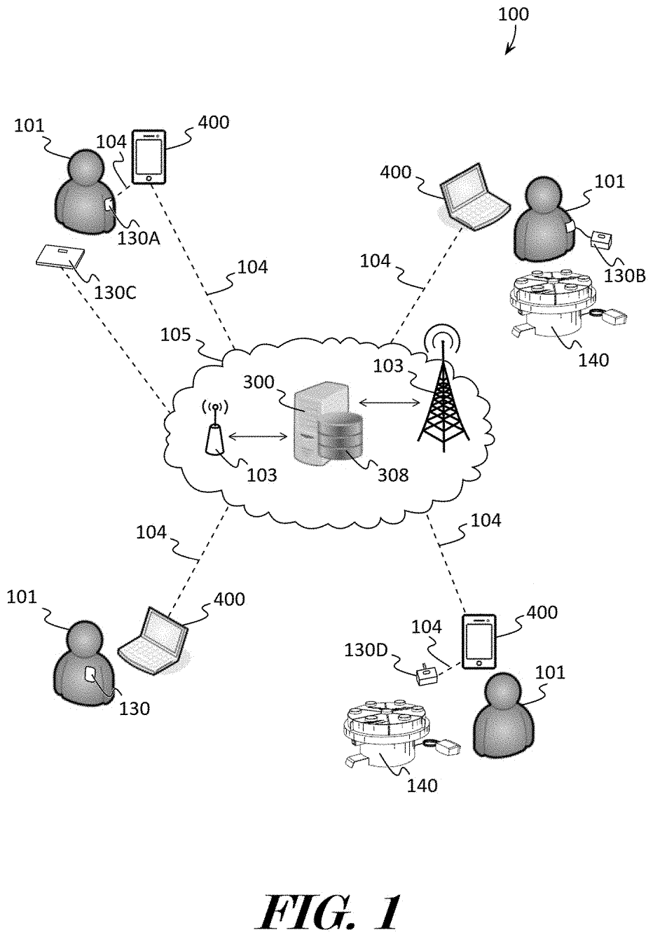

The present invention will now be described by example and through referencing the appended figures representing preferred and alternative embodiments. As perhaps best shown by FIG. 1, an illustrative example of some of the physical components which may comprise a system providing dietary and health information (“the system”) 100 according to some embodiments is presented. The system 100 is configured to facilitate the transfer of data and information between one or more health monitoring devices 130, dietary metering devices 140, access points 103, client devices 400, and servers 300 over a data network 105. A data store 308 accessible by the server 300 may contain one or more databases. Each client device 400 may send data to and receive data from the data network 105 through a network connection 104 with an access point 103.

The data may comprise any information that one or more users 101 desire to input into the system 100 including information describing one or more users 101, information describing the actions of one or more users 101, information requested by one or more users 101, information supplied by one or more users 101, dietary information, food and other edible nutrition information, health information, exercise information, health biometrics and test result information, and any other information which a user 101 may desire to input or enter into the system 100.

In this example, the system 100 comprises at least one client device 400 (but preferably more than two client devices 400) configured to be operated by one or more users 101. Client devices 400 can be mobile devices, such as laptops, tablet computers, personal digital assistants, smart phones, and the like, that are equipped with a wireless network interface capable of sending data to one or more servers 300 with access to one or more data stores 308 over a network 105 such as a wireless local area network (WLAN). Additionally, client devices 400 can be fixed devices, such as desktops, workstations, and the like, that are equipped with a wireless or wired network interface capable of sending data to one or more servers 300 with access to one or more data stores 308 over a wireless or wired local area network 105. The present invention may be implemented on at least one client device 400 and/or server 300 programmed to perform one or more of the steps described herein. In some embodiments, more than one client device 400 and/or server 300 may be used, with each being programmed to carry out one or more steps of a method or process described herein.

The system 100 may also comprise one or more health monitoring devices 130 which may be configured to be in wireless (such as Wi-Fi, Bluetooth, RFID, etc.) and/or wired communication with a dietary metering device 140, client device 400, and/or server 300. Generally, a health monitoring device 130 may comprise an electronic device that may be used to provide health biometric data to the system 100, including test result information, the biometric data describing one or more biometric parameters of a user's body 101.

In some embodiments, a health monitoring device 130 may comprise a glucose device 130A which may be an electronic patch that may be wearable by a user 101 having data connectivity functionality. The patch device 130A may be configured to provide health biometric information describing a user's 101 blood glucose levels, serum cholesterol levels, blood pressure readings, and/or any other health biometric and test result information of the user 101 which may be electronically communicated from the patch device 130A to a client device 400 and/or dietary metering device 140 via a wired or wireless connection 104. An example glucose device 130A includes the FreeStyle Libre by Abbott Diabetes Care.

In further embodiments, a health monitoring device 130 may comprise a blood pressure cuff/machine device 130B having data connectivity functionality. The blood pressure cuff/machine device 130B may be configured to provide health biometric information describing a user's 101 blood pressure and pulse which may be electronically communicated from the blood pressure cuff/machine device 130B to a client device 400 and/or dietary metering device 140 via a wired or wireless connection 104. An example blood pressure cuff/machine device 130B includes the Omron Wireless Blood Pressure Monitor by Omron.

In further embodiments, a health monitoring device 130 may comprise a weight scale device 130C having data connectivity functionality. The weight scale device 130C may be configured to provide health biometric information describing a user's 101 weight and optionally body fat percentage which may be electronically communicated from the weight scale device 130C to a client device 400 and/or dietary metering device 140 via a wired or wireless connection 104. An example weight scale device 130C includes the Garmin Index Smart Scale by Garmin.

In further embodiments, a health monitoring device 130 may comprise a standalone device 130D having data connectivity functionality. A standalone device 130D a serum cholesterol level measuring machine, a glucose test strip reading machine, or any other device that may not be wearable for extended periods of time but which may be used to periodically provide health biometric information of the user 101 that may be electronically communicated from the standalone device 130D to a client device 400 and/or dietary metering device 140 via a wired or wireless connection 104. An example standalone device 130D includes the OneTouch Verio Flex glucose meter by OneTouch and Smart Bluetooth Blood Glucose/Cholesterol Meter by Andesfit Health.

The system 100 may also comprise one or more dietary metering devices 140 which may be in communication with a health monitoring device 130, client device 400, and/or server 300. Generally, a dietary metering device 140 may comprise an electronic device which may be configured to store and dispense edible nutritional items. A user 101 may interact with the dietary metering device 140 to dispense metered amounts of the edible nutritional items which may then be consumed by the user 101.

The system 100 is configured to provide dietary health and information which may be utilized by a user to achieve healthier eating habits and exercise to lose weight and maintain good cholesterol, glucose, blood pressure, and sugar levels. The system 100 may receive biometric and diagnostic health information of the user 101, via a client device 400 or other device 120, 130, such as age, height, weight, glucose levels, blood pressure levels, and cholesterol levels. In preferred embodiments, the system 100 may receive biometric and diagnostic health information on a daily basis, and a health application 121 of the system 100 may calculate whether health results, such as weight, blood pressure, cholesterol levels, and glucose levels of the user 101 are improving. In further preferred embodiments, the health application 121 may provide suggestions to user 101, such as on a food plan, based on said results. In further preferred embodiments, the health application 121 may also periodically give trends in a graphical manner of the data for a user 101 to visually observe the actual results.

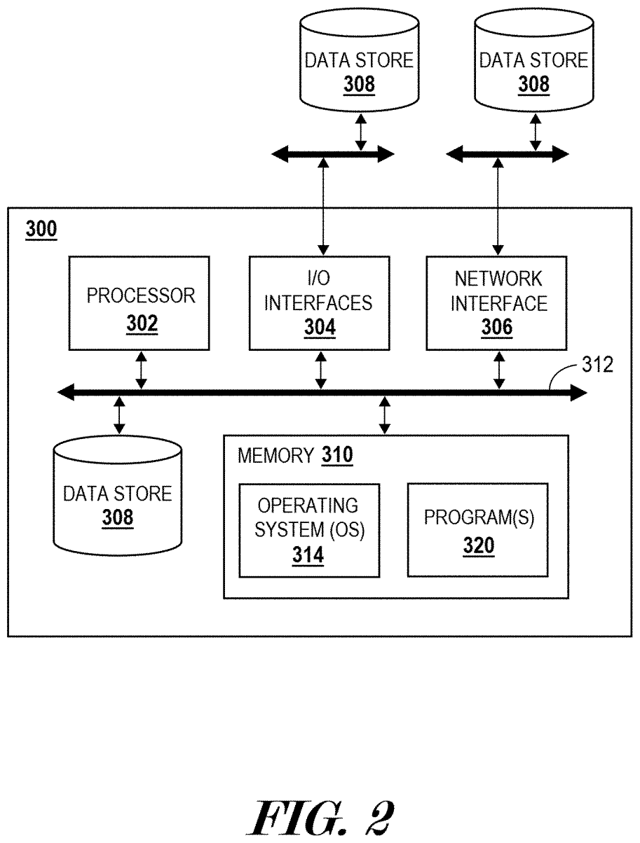

Referring now to FIG. 2, in an exemplary embodiment, a block diagram illustrates a server 300 of which one or more may be used in the system 100 or standalone. The server 300 may be a digital computer that, in terms of hardware architecture, generally includes a processor 302, input/output (I/O) interfaces 304, a network interface 306, a data store 308, and memory 310. It should be appreciated by those of ordinary skill in the art that FIG. 2 depicts the server 300 in an oversimplified manner, and a practical embodiment may include additional components and suitably configured processing logic to support known or conventional operating features that are not described in detail herein. The components (302, 304, 306, 308, and 310) are communicatively coupled via a local interface 312. The local interface 312 may be, for example but not limited to, one or more buses or other wired or wireless connections, as is known in the art. The local interface 312 may have additional elements, which are omitted for simplicity, such as controllers, buffers (caches), drivers, repeaters, and receivers, among many others, to enable communications. Further, the local interface 312 may include address, control, and/or data connections to enable appropriate communications among the aforementioned components.

The processor 302 is a hardware device for executing software instructions. The processor 302 may be any custom made or commercially available processor, a central processing unit (CPU), an auxiliary processor among several processors associated with the server 300, a semiconductor-based microprocessor (in the form of a microchip or chip set), or generally any device for executing software instructions. When the server 300 is in operation, the processor 302 is configured to execute software stored within the memory 310, to communicate data to and from the memory 310, and to generally control operations of the server 300 pursuant to the software instructions. The I/O interfaces 304 may be used to receive user input from and/or for providing system output to one or more devices or components. User input may be provided via, for example, a keyboard, touch pad, and/or a mouse. System output may be provided via a display device and a printer (not shown). I/O interfaces 304 may include, for example, a serial port, a parallel port, a small computer system interface (SCSI), a serial ATA (SATA), a fibre channel, Infiniband, iSCSI, a PCI Express interface (PCI-x), an infrared (IR) interface, a radio frequency (RF) interface, and/or a universal serial bus (USB) interface.

The network interface 306 may be used to enable the server 300 to communicate on a network, such as the Internet, the data network 105, the enterprise, and the like, etc. The network interface 306 may include, for example, an Ethernet card or adapter (e.g., 10BaseT, Fast Ethernet, Gigabit Ethernet, 10 GbE) or a wireless local area network (WLAN) card or adapter (e.g., 802.11a/b/g/n). The network interface 306 may include address, control, and/or data connections to enable appropriate communications on the network.

A data store 308 may be used to store data. The data store 308 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 308 may incorporate electronic, magnetic, optical, and/or other types of storage media. In one example, the data store 308 may be located internal to the server 300 such as, for example, an internal hard drive connected to the local interface 312 in the server 300. Additionally in another embodiment, the data store 308 may be located external to the server 300 such as, for example, an external hard drive connected to the I/O interfaces 304 (e.g., SCSI or USB connection). In a further embodiment, the data store 308 may be connected to the server 300 through a network, such as, for example, a network attached file server. Preferably, the system 100 may comprise a system database 330 which may be stored in one or more data stores 308.

The memory 310 may include any of volatile memory elements (e.g., random access memory (RAM, such as DRAM, SRAM, SDRAM, etc.)), nonvolatile memory elements (e.g., ROM, hard drive, tape, CDROM, etc.), and combinations thereof. Moreover, the memory 310 may incorporate electronic, magnetic, optical, and/or other types of storage media. Note that the memory 310 may have a distributed architecture, where various components are situated remotely from one another, but can be accessed by the processor 302. The software in memory 310 may include one or more software programs, each of which includes an ordered listing of executable instructions for implementing logical functions. The software in the memory 310 may include a suitable operating system (O/S) 314 and one or more programs 320.

The operating system 314 essentially controls the execution of other computer programs, such as the one or more programs 320, and provides scheduling, input-output control, file and data management, memory management, and communication control and related services. The operating system 314 may be, for example Windows NT, Windows 2000, Windows XP, Windows Vista, Windows 7, Windows 8, Windows 10, Windows Server 2003/2008/2012/2016 (all available from Microsoft, Corp. of Redmond, Wash.), Solaris (available from Sun Microsystems, Inc. of Palo Alto, Calif.), LINUX (or another UNIX variant) (available from Red Hat of Raleigh, N.C. and various other vendors), Android and variants thereof (available from Google, Inc. of Mountain View, Calif.), Apple OS X and variants thereof (available from Apple, Inc. of Cupertino, Calif.), or the like. The one or more programs 320 may be configured to implement the various processes, algorithms, methods, techniques, etc. described herein.

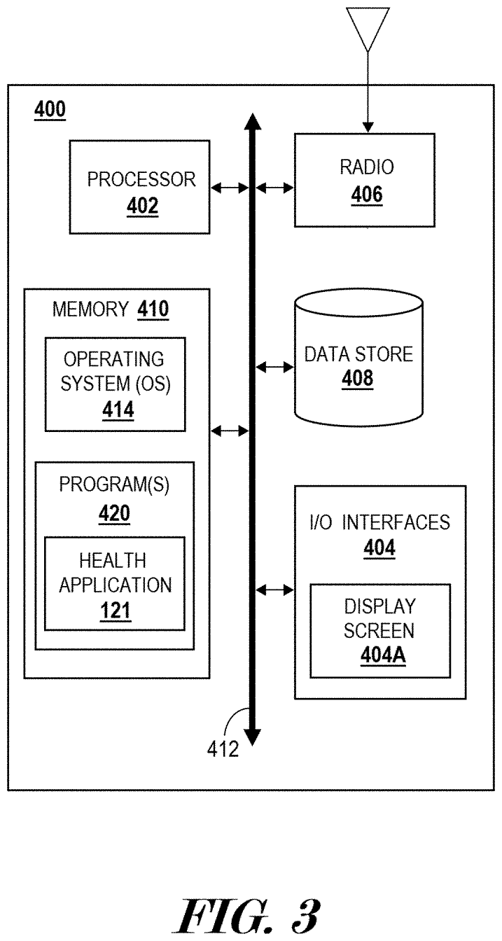

Referring to FIG. 3, in an exemplary embodiment, a block diagram illustrates a client device 400 of which one or more may be used in the system 100 or the like. The client device 400 can be a digital device that, in terms of hardware architecture, generally includes a processor 402, input/output (I/O) interfaces 404, a radio 406, a data store 408, and memory 410. It should be appreciated by those of ordinary skill in the art that FIG. 3 depicts the client device 400 in an oversimplified manner, and a practical embodiment may include additional components and suitably configured processing logic to support known or conventional operating features that are not described in detail herein. The components (402, 404, 406, 408, and 410) are communicatively coupled via a local interface 412. The local interface 412 can be, for example but not limited to, one or more buses or other wired or wireless connections, as is known in the art. The local interface 412 can have additional elements, which are omitted for simplicity, such as controllers, buffers (caches), drivers, repeaters, and receivers, among many others, to enable communications. Further, the local interface 412 may include address, control, and/or data connections to enable appropriate communications among the aforementioned components.

The processor 402 is a hardware device for executing software instructions. The processor 402 can be any custom made or commercially available processor, a central processing unit (CPU), an auxiliary processor among several processors associated with the client device 400, a semiconductor-based microprocessor (in the form of a microchip or chip set), or generally any device for executing software instructions. When the client device 400 is in operation, the processor 402 is configured to execute software stored within the memory 410, to communicate data to and from the memory 410, and to generally control operations of the client device 400 pursuant to the software instructions. In an exemplary embodiment, the processor 402 may include a mobile optimized processor such as optimized for power consumption and mobile applications.

The I/O interfaces 404 can be used to receive data and user input and/or for providing system output. User input can be provided via a plurality of I/O interfaces 404, such as a keypad, a touch screen, a camera, a microphone, a scroll ball, a scroll bar, buttons, bar code scanner, voice recognition, eye gesture, and the like. System output can be provided via a display device 404A, such as a liquid crystal display (LCD), light emitting diode (LED) display, touch screen display, and the like. The I/O interfaces 404 can also include, for example, a serial port, a parallel port, a small computer system interface (SCSI), an infrared (IR) interface, a radio frequency (RF) interface, a universal serial bus (USB) interface, and the like. The I/O interfaces 404 can include a graphical user interface (GUI) that enables a user to interact with the client device 400. Additionally, the I/O interfaces 404 may be used to output notifications to a user and can include a speaker or other sound emitting device configured to emit audio notifications, a vibrational device configured to vibrate, shake, or produce any other series of rapid and repeated movements to produce haptic notifications, and/or a light emitting diode (LED) or other light emitting element which may be configured to illuminate to provide a visual notification.

The radio 406 enables wireless communication to an external access device or network. Any number of suitable wireless data communication protocols, techniques, or methodologies can be supported by the radio 406, including, without limitation: RF; IrDA (infrared); Bluetooth; ZigBee (and other variants of the IEEE 802.15 protocol); IEEE 802.11 (any variation); Z-Wave wireless communications protocol used primarily for home automation; IEEE 802.16 (WiMAX or any other variation); Direct Sequence Spread Spectrum; Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication. The data store 408 may be used to store data. The data store 408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 408 may incorporate electronic, magnetic, optical, and/or other types of storage media.

The memory 410 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 410 may incorporate electronic, magnetic, optical, and/or other types of storage media. Note that the memory 410 may have a distributed architecture, where various components are situated remotely from one another, but can be accessed by the processor 402. The software in memory 410 can include one or more software programs, each of which includes an ordered listing of executable instructions for implementing logical functions. In the example of FIG. 3, the software in the memory system 410 includes a suitable operating system (O/S) 414 and programs 420.

The operating system 414 essentially controls the execution of other computer programs, and provides scheduling, input-output control, file and data management, memory management, and communication control and related services. The operating system 414 may be, for example, LINUX (or another UNIX variant), Android (available from Google), Symbian OS, Microsoft Windows CE, Microsoft Windows 7 Mobile, iOS (available from Apple, Inc.), webOS (available from Hewlett Packard), Blackberry OS (Available from Research in Motion), and the like. The programs 420 may include various applications, add-ons, etc. configured to provide end user functionality with the client device 400. For example, exemplary programs 420 may include, but not limited to, a web browser, social networking applications, streaming media applications, games, mapping and location applications, electronic mail applications, financial applications, and the like. In a typical example, the end user 101 typically uses one or more of the programs 420 along with a network 105 to exchange information with the system 100.

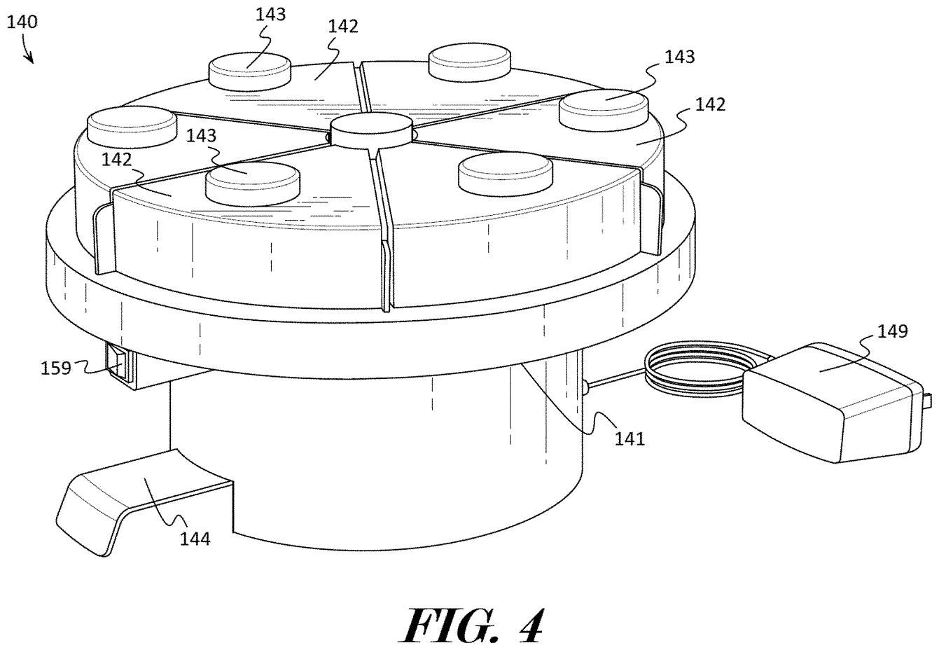

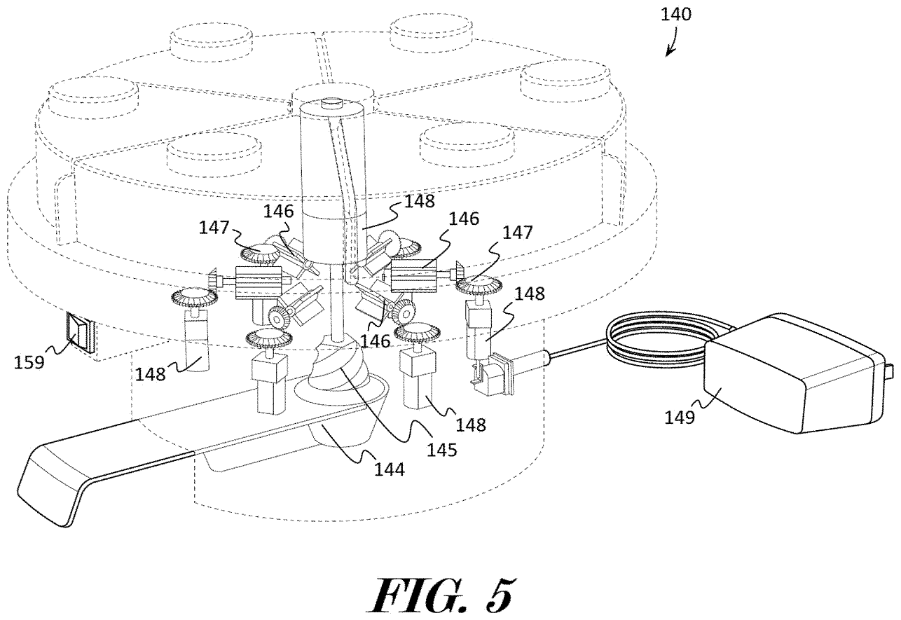

Referring now to FIGS. 4-6, and 9, examples of a dietary metering device (“the device”) 140 are illustrated according to various embodiments. The device 140 may comprise a housing 141 which may support and position one or more of the other elements of the device 140. A housing 141 may be configured in any shape and size. One or more, such as six, reservoirs 142 may be coupled to or otherwise supported by the housing 141. Each reservoir 142 may be configured to hold or contain a volume of an edible nutritional item. Reservoirs 142 may be configured in any size and shape to hold and desired volume of an edible nutritional item. Preferably, a lid 143 may be removably coupled to each reservoir 142 to govern access to the interior of the reservoir 142, such as to maintain the freshness of edible nutritional items within the reservoir 142.

An edible nutritional item may comprise any substance which may be eaten or otherwise ingested by a user 101. In preferred embodiments, nutritional items may comprise spices which typically include seed, fruit, root, bark, or other plant substances often used for flavoring, coloring or preserving food. Example spices include cardamom, cinnamon, allspice, cloves, nutmeg, pepper, turmeric, ginger, mace, saffron, vanilla, cumin, dill seed. In further embodiments, nutritional items may comprise herbs, which are the leaves, flowers, or stems of plants used for flavoring or as a garnish. Example herbs include basil, bay leaf, celery seed, chives, cilantro, dill, fennel, lemon grass, oregano, parsley, rosemary, sage, tarragon, and thyme. It should be understood, that any edible substance may be contained in a reservoir 142.

The device 140 may be configured to communicate metered amounts of edible nutritional items from each reservoir 142 into a receptacle 144. A receptacle 144 may be configured in any size and shape. Preferably, a receptacle 144 may be configured generally as a spoon having a bowl or the like for receiving various amounts of edible nutritional items.

In some embodiments, the device 140 may comprise a grinder 145. In preferred embodiments, edible nutritional items may be communicated from each reservoir 142 into a receptacle 144 upon passing through a grinder 145 which may reduce the size of the edible nutritional items. Optionally, a grinder 145 may be configured to grind the edible nutritional items in a range of sizes, such as between a course size, a fine size, and sizes between. A grinder 145 may comprise one or more blades, revolving abrasive surfaces, or any other suitable method or device for reducing the size of edible nutritional items.

In preferred embodiments, metered amounts of edible nutritional items may be communicated from each reservoir 142 into a grinder 145 via one or more dispensers 146, motivators 147, and motors 148. In some embodiments, a dispenser 146 may be generally configured as a paddle wheel so that by revolving the dispenser 146, one or more paddles may rotate to propel or otherwise dispense metered amounts of edible nutritional items. For example, each rotation of a dispenser 146 may propel or dispense approximately one gram of edible nutritional items from a reservoir 142 into a grinder 145. In other embodiments, a dispenser 146 may be configured as a conveyor belt, a screw pump, or any other method or device for propelling or otherwise dispensing metered amounts of edible nutritional items.

In some embodiments, a dispenser 146 may be moved by one or more motivators 147 and motors 148. A motivator 147 may comprise any mechanical arrangement which provides controlled application of power, such as a gearbox or the like that uses gears and gear trains to provide speed and torque conversions from a rotating power source to another device. Preferably, a motor 148 may comprise an electric motor such as a brushed DC motor, brushless DC motor, switched reluctance motor, universal motor, AC polyphase squirrel-cage or wound-rotor induction motor, AC SCIM split-phase capacitor-start motor, AC SCIM split-phase capacitor-run motor, AC SCIM split-phase auxiliary start winding motor, AC induction shaded-pole motor, wound-rotor synchronous motor, hysteresis motor, synchronous reluctance motor, pancake or axial rotor motor, stepper motor, or any other type of motor.

In some embodiments, the device 140 may comprise a power source 149 which may provide electrical power to any component that may require electrical power. In further embodiments, a power source 149 may comprise a power cord, optionally including a transformer. In still further embodiments, a power source 149 may comprise a battery, such as a lithium ion battery, nickel cadmium battery, alkaline battery, or any other suitable type of battery, a fuel cell, a capacitor, a super capacitor, or any other type of energy storing and/or electricity releasing device. In further embodiments, a power source 149 may comprise a kinetic or piezo electric battery charging device, a solar cell or photovoltaic cell, and/or inductive charging or wireless power receiver. In further embodiments, a power source 149 may comprise a power charging and distribution module which may be configured to control the recharging of the power source 149, discharging of the power source 149, and/or distribution of power to one or more components of the device 140 that may require electrical power.

In some embodiments, the device 140 may comprise one or more control inputs 159 that a user 101 may interact with, such as turnable control knobs, depressible button type switches, a key pad, slide type switches, rocker type switches, touch screen graphical user interfaces (GUI), or any other suitable input that may be used to modulate electricity between components or to otherwise control functions of the device 140. Generally, a user 101 may interact with a control input 159 to control functions of the device 140, such as: to power on/off the device; to select an amount of an edible nutritional item to be dispensed from a reservoir 142 into a receptacle 144; to select how course or fine to grind an edible nutritional item dispensed from a reservoir 142 into a receptacle 144; to select from which reservoir 142 an edible nutritional item is to be dispensed; to enter into a control unit 150 which edible nutritional item is in which reservoir 142; and any other user 101 controlled function.

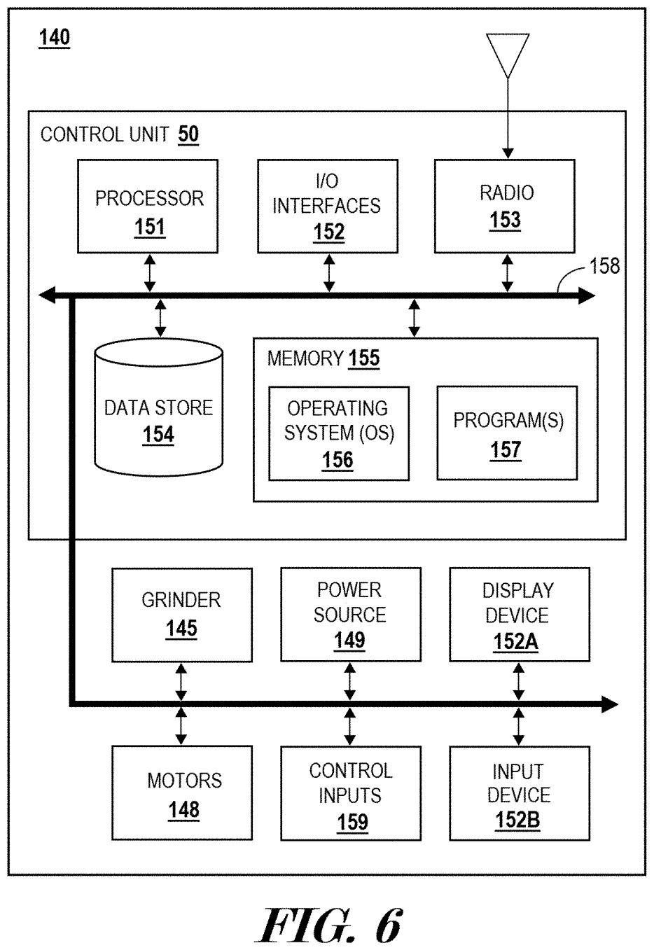

As perhaps best shown in FIG. 6, the device 140 may comprise a control unit 50 which may be in electronic communication with the motors 148, power source 149, control inputs 159, and any other element of the device 140. In some embodiments and in the present example, the device 140 can be a digital device that, in terms of hardware architecture, comprises a control unit 150 which optionally includes a processor 151, input/output (I/O) interfaces 152, a radio module 153, a data store 154, and memory 155. It should be appreciated by those of ordinary skill in the art that FIG. 6 depicts the device 140 in an oversimplified manner, and a practical embodiment may include additional components or elements and suitably configured processing logic to support known or conventional operating features that are not described in detail herein. The components and elements (150, 151, 152, 153, 154, 155, 148, 149, and 159) are communicatively coupled via a local interface 158. The local interface 158 can be, for example but not limited to, one or more buses, circuit boards, wiring harnesses, or other wired connections or wireless connections, as is known in the art. The local interface 158 can have additional elements, which are omitted for simplicity, such as controllers, buffers (caches), drivers, repeaters, and receivers, among many others, to enable communications. Further, the local interface 158 may include address, control, and/or data connections to enable appropriate communications among the aforementioned components.

The processor 151 is a hardware device for executing software instructions. The processor 151 can be any custom made or commercially available processor, a central processing unit (CPU), an auxiliary processor among several processors associated with the control unit 150, a semiconductor-based microprocessor (in the form of a microchip or chip set), or generally any device for executing software instructions. When the processing unit 150 is in operation, the processor 151 is configured to execute software stored within the memory 155, to communicate data to and from the memory 155, and to generally control operations of the device 140 pursuant to the software instructions and/or from instructions received from a control input 159. In an exemplary embodiment, the processor 51 may include a mobile optimized processor such as optimized for power consumption and mobile applications.

In addition to control inputs 159, the I/O interfaces 152 can be used to by a user 101 to provide input, such as which may be used to control one or more functions of the device 140. The I/O interfaces 152 can include, for example, buttons, knobs, switches, LED indicator lights, LED display, LCD display, a serial port, a parallel port, a small computer system interface (SCSI), an infrared (IR) interface, a radio frequency (RF) interface, a universal serial bus (USB) interface, and the like.

An optional radio module 153 enables wireless communication to an external access device or network. In preferred embodiments, a radio 153 may operate via WiFi communication standards. In further embodiments, a radio 153 may operate on a cellular band and may communicate with or receive a Subscriber Identity Module (SIM) card or other wireless network identifier. Any number of suitable wireless data communication protocols, techniques, or methodologies can be supported by the radio 153, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Near-Field Communication (NFC); Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication. An optional data store 154 may be used to store data. The data store 154 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 154 may incorporate electronic, magnetic, optical, and/or other types of storage media.

The memory 155 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 155 may incorporate electronic, magnetic, optical, and/or other types of storage media. Note that the memory 155 may have a distributed architecture, where various components are situated remotely from one another, but can be accessed by the processor 151. The software in memory 155 can include one or more software programs, each of which includes an ordered listing of executable instructions for implementing logical functions. In the example of FIG. 6, the software in the memory system 155 includes a suitable operating system (O/S) 156 and program(s) 157. The operating system 156 essentially controls the execution of input/output interface 152 functions, and provides scheduling, input-output control, file and data management, memory management, and communication control and related services. The operating system 156 may be, for example, LINUX (or another UNIX variant), Android (available from Google), Symbian OS, Microsoft Windows CE, Microsoft Windows 7 Mobile, iOS (available from Apple, Inc.), webOS (available from Hewlett Packard), Blackberry OS (Available from Research in Motion), and the like. The programs 157 may include various applications, add-ons, etc. configured to provide end user functionality with the device 140. For example, exemplary programs 157 may include, but not limited to, instructions for operating the motors 148 to dispense a metered amount of an edible nutritional item from one or more reservoirs 142. In a further example, a program 157 may enable one or more functions of the device 140 to be controlled via a health application 121 or other programs 320, 420, of one or more client devices 400 and/or servers 300.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The control unit 50 may also include a main memory, such as a random access memory (RAM) or other dynamic storage device (e.g., dynamic RAM (DRAM), static RAM (SRAM), and synchronous DRAM (SDRAM)), coupled to the bus for storing information and instructions to be executed by the processor 51. In addition, the main memory may be used for storing temporary variables or other intermediate information during the execution of instructions by the processor 51. The control unit 50 may further include a read only memory (ROM) or other static storage device (e.g., programmable ROM (PROM), erasable PROM (EPROM), and electrically erasable PROM (EEPROM)) coupled to the bus for storing static information and instructions for the processor 51.

While some exemplary shapes and sizes have been provided for elements of the device 140, it should be understood to one of ordinary skill in the art that the housing 11, reservoirs 142, receptacle 144, and any other element described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Additionally, while some materials have been provided, in other embodiments, the elements that comprise the device 140 may be made from or may comprise durable materials such as aluminum, steel, other metals and metal alloys, wood, hard rubbers, hard plastics, fiber reinforced plastics, carbon fiber, fiber glass, resins, polymers or any other suitable materials including combinations of materials. Additionally, one or more elements may be made from or comprise durable and slightly flexible materials such as soft plastics, silicone, soft rubbers, or any other suitable materials including combinations of materials. In some embodiments, one or more of the elements that comprise the device 140 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 140 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 140 may be coupled by being one of connected to and integrally formed with another element of the device 140.

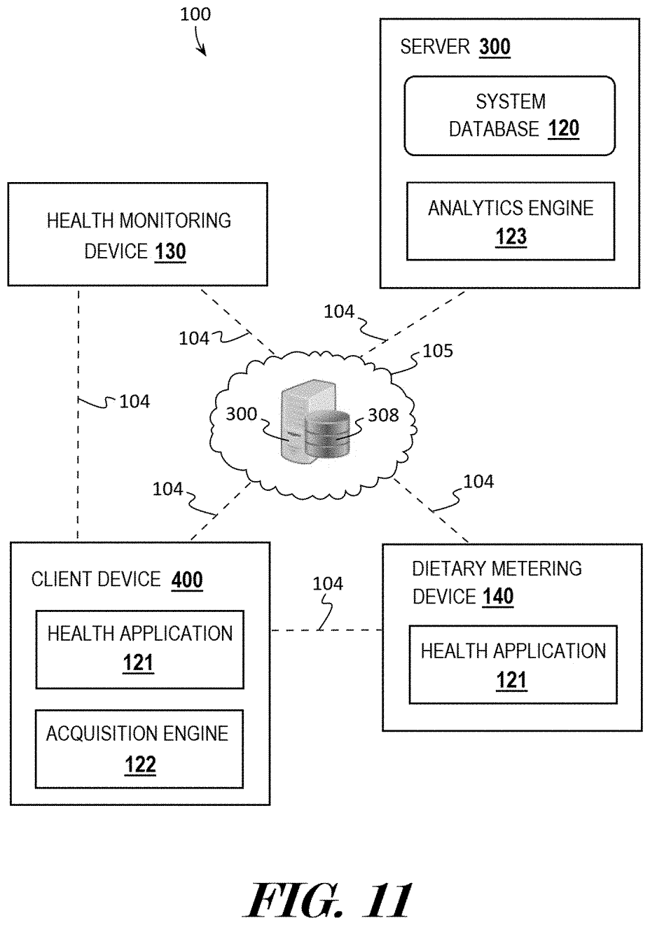

Referring now to FIG. 11 a block diagram showing some software rules engines and components which may be found in a system 100 and which may optionally be configured to run on one or more dietary metering device 140, servers 300, and/or client devices 400 according to various embodiments described herein are illustrated. A dietary metering device 140, server 300, and/or client device 400 may be in wired and/or wireless electronic communication through a network 105 with a data store 308. The engines 121, 122, 123, may be in electronic communication so that data may be readily exchanged between the engines 121, 122, 123, and one or more engines 121, 122, 123, may read, write, or otherwise access data in one or more databases 120 of one or more data stores 308.

In this and some embodiments, one or more servers 300 may be configured to run one or more software rules engines or programs such as an analytics engine 123, while a client device 400 and/or a health monitoring device 130 may be configured to run one or more software rules engines or programs such as a health application 121 and an acquisition engine 122. In other embodiments, a health application 121, an acquisition engine 122, and/or an analytics engine 123 may be configured to run on one or more dietary metering devices 140, client devices 400, and/or servers 300 with data transferred to and from a health application 121, an acquisition engine 122, and/or an analytics engine 123 that may be in communication with a data store 308 through a network 105. It should be understood that the functions attributed to the engines 121, 122, 123, described herein are exemplary in nature, and that in alternative embodiments, any function attributed to any engine 121, 122, 123, may be performed by one or more other engines 121, 122, 123, or any other suitable processor logic.

The system 100 may comprise one or more databases, such as a system database 120, which may be stored on a data store 308 accessible to one or more engines 121, 122, 123. It should be understood that the described structure of the system database 120 is exemplary in nature, and that in alternative embodiments, the data contained within the system database 120 may be organized in any other way.

In some embodiments, a system database 120 may comprise information that one or more users 101, such as a plurality of users 101, desire to input into the system 100 including information provided by one or more users 101, such as information on or describing one or more users 101, baseline health information of each user 101, medical test results, other medical diagnostic results of one or more users 101, information on or describing one or more medical conditions and diagnoses, information on or describing dietary habits of one or more users 101, information on or describing dietary allergies of one or more users 101, information on or describing activity or exercise habits of one or more users 101, and any other information which a user 101 may provide or be provided with for the purposes of receiving dietary and health information.

In some embodiments, a system database 120 may also include nutrition and exercise data for each user 101. Nutrition data may include data describing the types of foods and drinks each user 101 consumes, the amounts of foods and drinks each user 101 consumes, the times each user 101 consumes foods and drinks throughout the day, and any other data which may describe the caloric food and drink intake (and non-caloric intake, such as water) of each user 101. The system database 120 may also include a calorie count table that describes the amount of calories in one or more volumes of foods, including in edible nutritional items. Exercise data may include data describing the types of physical activities and exercise, amounts of physical activities and exercise, and times of physical activities and exercise that each user 101 participates in. In further embodiments, a system database 120 may comprise biometric data for each user 101 that may be recorded by one or more health monitoring devices 130. Example biometric data for each user 101 may comprise one or more weight measurements, cholesterol measurements, blood pressure measurements, and blood glucose measurements.

In some embodiments, a system database 120 may also include one or more modifications which may be provided to a user 101. Generally, a modification may comprise a health recommendation that the system 100 may provide to a user 101, and the health recommendation may include information describing an exercise recommendation that a user 101 may perform to enable the user 101 to achieve one or more biometric data goals and/or the health recommendation may include information describing a dietary recommendation that a user 101 may perform to enable the user 101 to achieve one or more biometric data goals. As an example, an exercise recommendation may comprise information describing a cardio fitness exercise, such as jogging, and how long and/or far the user 101 should jog and how frequently the user should jog. As another example, a dietary recommendation may comprise a minimum number of food calories, a maximum number of food calories, and/or a range of food calories that a user 101 should consume in a day or at a meal/snack time. In preferred embodiments, a dietary recommendation may comprise an amount of an edible nutritional item that may be stored in and dispensed from a dietary metering device 140 that a user 101 may consume to meet a biometric goal. Generally, modifications may describe dietary and/or exercise changes that may be implemented to the nutrition and exercise routine of a user 101 that when implemented may result in the user 101 meeting their biometric goal(s).

The system 100 may comprise one or more health applications 121. A health application 121 may comprise or function as communication logic stored in a memory 310, 410, 155, which may be executable by the processor 302, 402, 151, of a server 300, client device 400, and/or dietary metering device 140. Generally, a health application 121 may be run on a client device 400, and/or dietary metering device 140 to provide user interface for a user to input data to the system 100 and for outputting system data to a user 101.

In some embodiments, a health application 121 may be configured to operate an I/O interface 404, such as a display screen 404A (optionally of a touchscreen interface), of a client device 400 operated by a user 101 in order to provide and receive information from the user 101. In preferred embodiments, a health application 121 may be configured to provide one or more modifications to the user 101, via their client device 400, as selected by an analytics engine 123. In further embodiments, a health application 121 may be configured to receive input from a user 101, in which the input comprises a biometric data goal, via an I/O interface 404 of a client device 400. In further embodiments, a health application 121 may be configured to receive input from a user 101, in which the input comprises baseline health information of a user 101, via an I/O interface 404 of a client device 400. In still further embodiments, a communication engine 132 may be configured to operate any of the I/O interfaces 404 of a client device 400 to allow the system 100 to input and output information from and to a user 101 via a client device 400.

The system 100 may comprise one or more acquisition engines 122. An acquisition engine 122 may comprise or function as acquisition logic stored in a memory 310, 410, which may be executable by the processor 302, 402, of a server 300 and/or client device 400. Generally, an acquisition engine 122 may be run on a client device 400, and/or server 300 to receive user data, user biometric data, user nutrition data, and user exercise data from health monitoring devices 130 and client devices 400, and to provide the data to an analytics engine 123.

In some embodiments, an acquisition engine 122 may acquire or receive baseline health information of a user 101 from health monitoring devices 130, dietary metering devices 140, and/or client devices 400 and then communicate the baseline health information to an analytics engine 123. In this manner, an acquisition engine 122 be configured to interface with a plurality of different brands monitoring devices 130, dietary metering devices 140, and/or client devices 400 to allow the analytics engine 123 to receive and send data with these devices 130, 140, 400. In further embodiments an acquisition engine 122 may acquire or receive nutrition or food intake data of a user, such as by using Radio-frequency identification (RFID) tags embedded in a food container read by dietary metering devices 140, and/or client devices 400, and by using a camera to scan the UPC code of food items, via dietary metering devices 140, and/or client devices 400. In further embodiments an acquisition engine 122 may acquire or receive exercise data from a health application 121 of a server 300, client device 400, and/or dietary metering device 140.

The system 100 may comprise one or more analytics engines 123. An analytics engine 123 may comprise or function as analytic logic stored in a memory 310, 410, which may be executable by the processor 302, 402, of a server 300 and/or client device 400. Preferably, an analytics engine 123 may be run on a server 300 to analyze user data, user biometric data, user nutrition data, and user exercise data from health monitoring devices 130 and client devices 400, and to generate modifications for the users 101 of the system 100.

An analytics engine 123 may be configured to generate one or more modifications for the users 101 of the system 100. Generally, a modification may comprise a health recommendation that the system 100 may provide to a user 101. A health recommendation may include information describing an activity that a user 101 may perform, and by performing that activity, the health of the user 101 may be improved to enable the user 101 to achieve one or more biometric data goals that they have input into the system 100. In some embodiments, a health recommendation may comprise or include an exercise recommendation that a user 101 may perform to enable the user 101 to achieve one or more biometric data goals. In some embodiments, a health recommendation may comprise or include a health recommendation may include information describing a dietary recommendation that a user 101 may perform to enable the user 101 to achieve one or more biometric data goals.

In some embodiments, a biometric data goal of a user 101 may describe a desired change in the weight of the user 101, and analytics engine 123 may provide a dietary recommendation, to the health application 121 running on a client device 400 or dietary metering device 140 that describes a daily number of calories that the user 101 should consume to enable the user 101 to achieve the desired change in the weight of the user 101.

In some embodiments, an analytics engine 123 may be configured to analyze the user data, user biometric data, user nutrition data, and user exercise data of a single user 101 and based on this data (single user data) the analytics engine 123 may generate one or more modifications for that user 101. For example, an analytics engine 123 may be configured to: determine effect of exercise data of a user 101 on the biometric data of that user 101; determine effect of food intake data of a user 101 on biometric data of that user 101; analyze exercise data of a user 101 and analyze the type and amount of exercises and its effect on the body's weight, cholesterol, blood pressure, and glucose levels of that user 101; analyze food intake data of a user 101 and analyze the type of food they are eating and its effect on the body's weight, cholesterol, blood pressure, and glucose levels of that user 101; and analyze biometric data of a user 101 as a whole. Using this single user data, the analytics engine 123 may then generate one or more recommendations for the user 101, which when performed by the user 101 will improve their health. As examples, an analytics engine 123 using single user data may: provide a health recommendation having a dietary recommendation that informs the user 101 what food to eat to stabilize your weight, cholesterol, blood pressure, and glucose levels (preferably may pull up the food suggestions based on an embedded calorie count table); provide a health recommendation having a dietary recommendation that provides preferable food options to the user 101 not simply based on calorie count, but rather based on an entire user profile from blood levels, sugar levels, cholesterol levels, etc., attained from their respective devices; and provide a health recommendation having an exercise recommendation which includes exercises to perform to stabilize the user's weight, cholesterol, blood pressure, and glucose levels.

In further embodiments, an analytics engine 123 may be configured to analyze the user data, user biometric data, user nutrition data, and user exercise data of a two or more, and more preferably, a plurality of users 101 and based on this data the analytics engine 123 may generate one or more modifications for a user 101. For example, an analytics engine 123 may be configured to: determine effect of exercise data of a plurality of users 101 on the biometric data of the plurality of users 101; determine effect of food intake data of a plurality of users 101 on biometric data of the plurality of users 101; analyze exercise data of a plurality of users 101 and analyze the type and amount of exercises and its effect on the body's weight, cholesterol, blood pressure, and glucose levels of the plurality of users 101; analyze food intake data of a plurality of users 101 and analyze the type of food they are eating and its effect on the body's weight, cholesterol, blood pressure, and glucose levels of the plurality of users 101; and analyze biometric data of the plurality of users 101 as a whole. Using this plurality user data, the analytics engine 123 may then generate one or more recommendations for the individual users 101 of the system 100, which when performed by a user 101 will improve their health. As examples, an analytics engine 123, using of the plurality of users 101 may: provide a health recommendation having a dietary recommendation that informs a user 101 what food to eat to stabilize their weight, cholesterol, blood pressure, and glucose levels (preferably may pull up the food suggestions based on an embedded calorie count table); provide a health recommendation having a dietary recommendation that provides preferable food options to a user 101 not simply based on calorie count, but rather based on an entire user profile from blood levels, sugar levels, cholesterol levels, etc., attained from their respective health monitoring devices 130; provide a health recommendation having an exercise recommendation which includes exercises to perform to stabilize a user's 101 weight, cholesterol, blood pressure, and glucose levels.

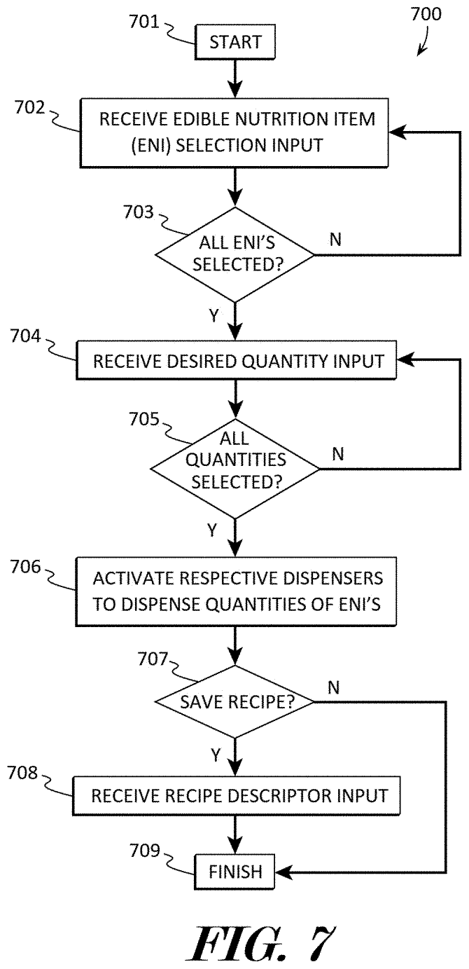

FIG. 7 illustrates a block diagram of an example of a method for dispensing edible nutritional items from a dietary metering device (“the method”) 700 according to various embodiments described herein. The method 700 may be used to dispense edible nutritional items from a dietary metering device 140 by a user 101. One or more steps of the method 700 may be performed by one or more health applications 121, acquisition engines 122, and/or an analytics engines 123 which may be executed by a computing device processor, such as a processor 151 (FIG. 6), processor 302 (FIG. 2), and/or a processor 402 (FIG. 3). It should be understood that the ordering of the steps may be modified while still enabling completion of the method 700.

The method 700 may start 701 and edible nutrition item selection input may be received in step 702. In some embodiments, the edible nutrition item selection input may be received from a user 101 via control input(s) 159 by a program 157 of a device 140. In further embodiments, edible nutrition item selection input may be received from a user 101 via a health application 121 running on a client device 400 and/or server 300. Generally, the edible nutrition item selection input may describe an edible nutrition item that the user 101 desires to have dispensed from a reservoir 142 and into a receptacle 144. Optionally, the edible nutrition item selection input may describe how the user 101 would like the edible nutrition item to be ground by a grinder 145.

In decision block 703, the device 140 may determine if all the desired edible nutrition items have been selected. In some embodiments, a program 157 of the device 140 may determine if all the desired edible nutrition items have been selected via input received from a user 101 via control input(s) 159 of a device 140. In further embodiments, a program 157 of the device 140 may determine if all the desired edible nutrition items have been selected via input received from a user 101 via a health application 121 running on a client device 400 and/or server 300. If all the desired edible nutrition items have not been selected, the method 700 may continue to step 702 to receive additional edible nutrition item selection input. If all the desired edible nutrition items have been selected, the method 700 may continue to step 704.

In step 704, desired quantity input may be received. In some embodiments, the desired quantity input may be received from a user 101 via control input(s) 159 by a program 157 of a device 140. In further embodiments, desired quantity input may be received from a user 101 via a health application 121 running on a client device 400 and/or server 300. Generally, the desired quantity input may describe the quantity of one or more edible nutrition items that the user 101 desires to have dispensed from a reservoir 142 and into a receptacle 144. Optionally, the desired quantity input may describe how the user 101 would like the edible nutrition item to be ground by a grinder 145.

In decision block 705, the device 140 may determine if all quantities of the desired edible nutrition items have been selected. In some embodiments, a program 157 of the device 140 may determine if all quantities of the desired edible nutrition items have been selected via input received from a user 101 via control input(s) 159 of a device 140. In further embodiments, a program 157 of the device 140 may determine if all quantities of the desired edible nutrition items have been selected via input received from a user 101 via a health application 121 running on a client device 400 and/or server 300. If all the quantities have not been selected, the method 700 may continue to step 704 to receive additional quantity selection input. If all the quantities of the desired edible nutrition items have been selected, the method 700 may continue to step 706.

In step 706, the dispensers 146 of the reservoirs 142 holding the desired edible nutrition items may be activated to dispense the desired quantities of the edible nutrition items. In some embodiments, a program 157 of the device 140 may active the motors 148 of the dispensers 146 to dispense the desired quantities of the edible nutrition items from the reservoirs 142 into the receptacle 144 preferably via a grinder 145.

In decision block 707, the device 140 may determine if the dispensed quantities of the edible nutrition items may be saved as a recipe, such as in a data store 154 of the device 140, a data store 408 of a client device 400, and/or a data store 308 of a server 300 for future access. In some embodiments, a program 157 of the device 140 may determine if the dispensed quantities of the edible nutrition items may be saved as a recipe via input received from a user 101 via control input(s) 159 of a device 140. In further embodiments, a program 157 of the device 140 may determine if the dispensed quantities of the edible nutrition items may be saved as a recipe via input received from a user 101 via a health application 121 running on a client device 400 and/or server 300. If the dispensed quantities of the edible nutrition items are not to be saved as a recipe, the method 700 may finish 709. If the dispensed quantities of the edible nutrition items are to be saved as a recipe, the method 700 may continue to step 708.

In step 708, recipe descriptor input may be received. In some embodiments, the recipe descriptor input may be received from a user 101 via control input(s) 159 by a program 157 of a device 140. In further embodiments, recipe descriptor input may be received from a user 101 via a health application 121 running on a client device 400 and/or server 300. Generally, the recipe descriptor input may describe the quantities of each edible nutrition items that were dispensed from a reservoir 142 and into a receptacle 144 in steps 702-707 for access at a later time. After step 708, the method 700 may finish 709.

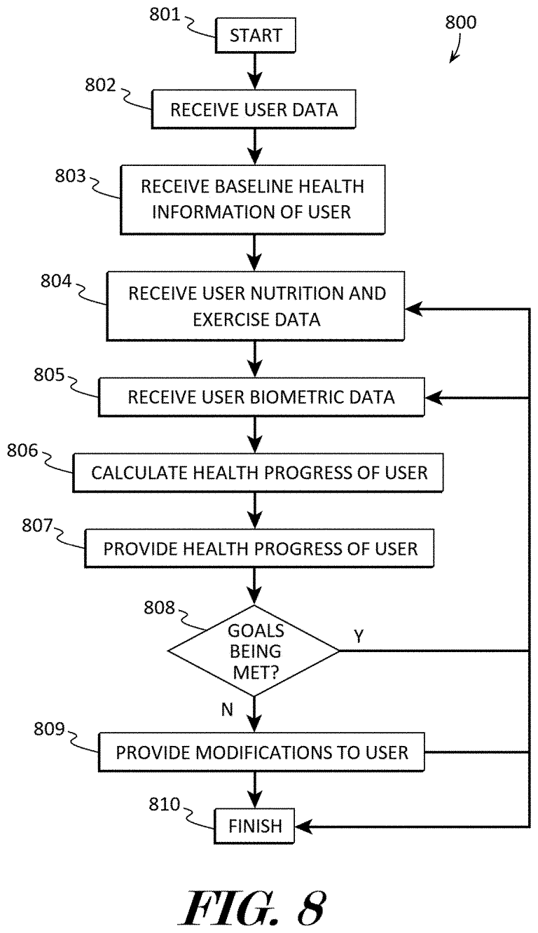

FIG. 8 shows a block diagram of an example of a method for providing dietary health and progress information (“the method”) 800 according to various embodiments described herein. The method 800 may be used to provide dietary health and progress information to a user 101 via a client device 400 and/or a dietary metering device 140. One or more steps of the method 800 may be performed by one or more health applications 121, acquisition engines 122, and/or an analytics engines 123 which may be executed by a computing device processor, such as a processor 151 (FIG. 6), processor 302 (FIG. 2), and/or a processor 402 (FIG. 3). It should be understood that the ordering of the steps may be modified while still enabling completion of the method 800.

The method 800 may start 801 and user data of a user 101 may be received from one or more health monitoring device(s) 130 and/or client devices 400 in step 802 via user 101 input provided to a health application 121. In some embodiments, user data 101 may include describe one or more health goals or biometric data goals of the user 101. For example, a biometric data goal may describe a desired body fat percentage, physical activity level, daily caloric intake, and other goals that the user 101 desires to achieve. Other example biometric data goals include: a desired body weight measurement or range of measurements, a desired cholesterol measurement or range of measurements, a desired blood pressure measurement or range of measurements, and a desired blood glucose measurement or range of measurements. In further embodiments, user data may describe one or more client devices 400, health monitoring devices 130, and/or dietary metering devices 140 that the user 101 desires to use. In still further embodiments, user data may include login credentials, billing information, contact information, and other account information for that user 101 which may be used by the system 100.

In step 803, baseline health information of the user 101 may be received. In some embodiments, the baseline health information may be received from a user 101 via control input(s) 159 by a health application 121 of a device 140. In further embodiments, baseline health information may be received from a user 101 via a health application 121 running on a client device 400 and/or server 300. Generally, the baseline health information may describe the baseline health information of the user 101, such as age, height, weight, glucose levels, blood pressure levels, and cholesterol levels. Preferably, baseline health information may be provided via one or more health monitoring devices 130. In some embodiments, a health monitoring device 130 may comprise a patch that may be wearable by a user 101, and which may be configured to provide information describing a user's 101 blood glucose levels, serum cholesterol levels, blood pressure readings, and/or any other health biometric and test result information of the user 101. In further embodiments, a health monitoring device 130 may comprise an electronic device or manually operated device, such as a blood pressure cuff/machine, a blood glucose strip testing device, a serum cholesterol level measuring machine, or any other device that may not be wearable for extended periods of time but which may be used to periodically provide health biometric and test result information of the user 101. Optionally, based on the baseline health information the health application 121 may provide daily calorie requirements for the user 101 via a display screen of the user's client device 400.

In step 804, user nutrition and exercise data of the user 101 may be received from one or more health monitoring device(s) 130 and/or client devices 400 via user 101 input provided to a health application 121. In some embodiments, the nutrition and exercise data may describe calorie count data and nutritional data of foods eaten by the user 101 over a period of time, such as a breakfast time period, lunch time period, snack time period, dinner time period, and daily time period, which may be entered by the user 101 into a health application 121 running on their client device 400 and/or server 300. In further embodiments, the nutrition and exercise data may comprise data recorded by the one or more health monitoring devices 130 utilized by the user 101. In still further embodiments, the user data may comprise data describing exercise and physical activity of the user 101 over a period of time.

In step 805, biometric data of the user 101 may be received. In some embodiments, an acquisition engine 122 may receive user biometric data from a health monitoring device 130, such as a patch device 130A, blood pressure cuff/machine device 130B, weight scale device 130C, and a standalone device 130D. The biometric data of the user 101 may include a body weight measurement, a cholesterol measurement, a blood pressure measurement, a blood glucose measurement, body fat percentage measurement, or any other human body metric.

In step 806, the health progress of the user 101 may be calculated. Generally, a health application 121 may use the information obtained in step 803 to calculate the resulting changes to the body and health of the user 101. For example, the health progress may be calculated using calorie intake and exercise statistics for the user 101 over a time period, such as a day, and how the user's weight may change if the calorie intake and exercise statistics are maintained and/or changed.

In step 807, the health progress of the user 101 may be provided. Health progress includes data describing changes of the user's 101 biometric data, and more preferably data describing the progress of a biometric data of the user 101 towards a biometric data goal of that user 101. In some embodiments, the health progress of the user 101 may be provided via a display screen 404A of a client device 400 and/or a display device 152A of a dietary metering device 140 running the health application 121 which is being utilized by the user 101. Optionally, the health progress of the user 101 may be provided to be shared with one or more other client devices 400, such as by being posted to social media sites and other social digital content sites.

In decision block 808, the health application 121 may determine if the goals of the user 101 are being met. In some embodiments, the health application 121 may determine if the goals of the user 101 entered in step 802 will be met using the health progress of the user that was calculated in step 806. If the goals of the user 101 are being met, the method 800 may cycle to step 804 and/or 805 any number of times and then on to step 806. If the goals of the user 101 are being met, the method 800 may proceed to step 809.

In step 809, modifications may be provided to the user 101. Generally, modifications may describe dietary and/or exercise changes that may be implemented to the nutrition and exercise data of step 804 that when implemented may result in the user 101 meeting their biometric data goals. Preferably, a health application 121 may provide the modifications via the user's client device 400 and optionally via a dietary metering device 140. After step 809, the method 800 may cycle to step 804 and/or 805 any number of times and then on to step 806 or optionally finish 810.

It should be understood that steps 804 and/or 805 may be performed any number of times to track the progress of a user 101 in achieving one or more of their biometric data goals. As another example of an implementation of method 800, input may be received from a user 101 through a health application 121 running on a client device 400, and the input may comprise one or more biometric data goals in step 802. In step 803, baseline health information of the user 101 may be received by an acquisition engine 122 via a health monitoring device 130, and the baseline health information may include a first biometric data in which the first biometric data is wirelessly or wiredly communicated from the health monitoring device 130 to the health application 121 on the client device 400 in step 803. In step 804, user nutrition and exercise data may be received via the health application 121 running on the client device 400. A first modification may be generated by an analytics engine 123 and provided to the user 101 via the health application 121 running on the client device, and the first modification may include a health recommendation to achieve the biometric data goal of the user 101. Then the method 800 may continue to step 805 in which a second biometric data may be taken after a period of time after the first biometric data is received. The method 800 may then continue from step 805 to steps 806, 807, 808, and then to step 809, so that the method 800 further comprises the step of providing a second modification to the user 101 via the client device, in which the second modification is provided after the step of recording the second biometric data.

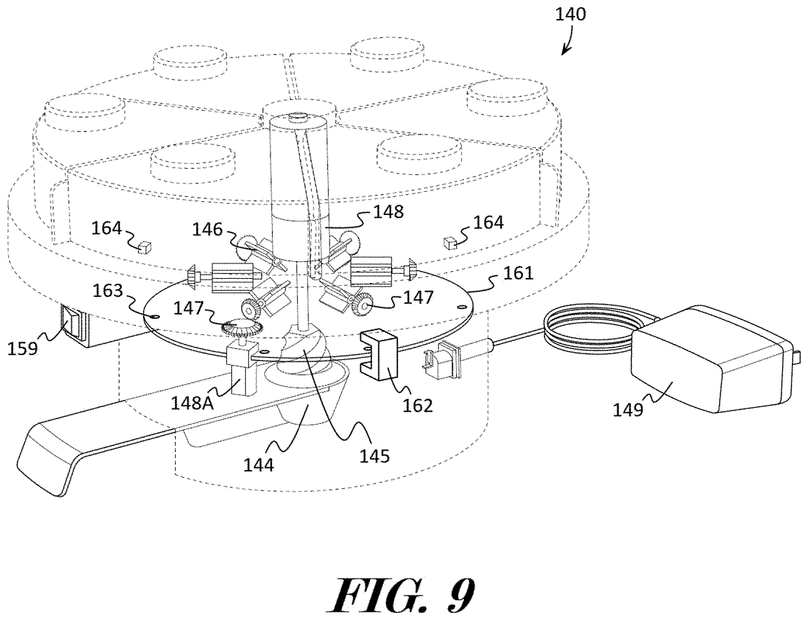

Turning now to FIG. 9, another embodiment of a dietary metering device 140 is depicted. In some embodiments, the device 140 may comprise a dispensing motor 148A which may be configured to be moved into various positions in order to operate the dispensers 146 of the device 140. Preferably, each reservoir 142 may be in communication with its own dispenser 146 and motivator 147. The dispensing motor 148A may be coupled to a carousel 161 which may be moved by a motor 148 which may move the carousel 161 in order to move the dispensing motor 148A into contact with a motivator 147 of a desired reservoir 142. Once in contact with a motivator 147 of a desired reservoir 142, the dispensing motor 148A may be actuated to cause the motivator 147 to dispense a metered amount of an edible nutritional item from the reservoir 142.

In preferred embodiments, a carousel 161 may be generally disk or cylinder shaped. In other embodiments, a carousel 161 may be configured in any other shape. In some embodiments, the device 140 may comprise a positional sensor 162 which may be in communication with the control unit 150, and data from the positional sensor 162 may be used by the control unit 150 to determine the position of the carousel 161 and therefore the position of the dispensing motor 148A. In preferred embodiments, a positional sensor 162 may comprise an optical sensor which may read one or more positional indicators 163 coupled to, formed into, or applied to the carousel 161. For example, a positional sensor 162 and carousel 161 with positional indicators 163 may comprise a light chopper wheel (rotatable by a motor 148 controlled by the control unit 150) and optical slot sensor or beam splitter. Light passing through or being blocked by the positional indicators 163 may be read by the optical positional sensor 162 in order to determine the position of the carousel 161/dispensing motor 148A. In other embodiments, a positional sensor 162 may comprise any other type of sensor which may be used to communicate positional data of the carousel 161/dispensing motor 148A to the control unit 150.

In preferred embodiments, each reservoir 142 of the device 140 may comprise a data device 164, such as a one-time programmable microcontroller, RFID tag, barcode, etc., which may be integrated into bottom or other location on each reservoir 142. The microcontroller, RFID tag, or other data device may comprise data which may be readable by the control unit 150. This data may include type of the edible nutritional item in a reservoir 142, date of manufacture of the edible nutritional item in a reservoir 142, keys verifying genuine product, and/or any other data which may describe the contents of the reservoir 142.

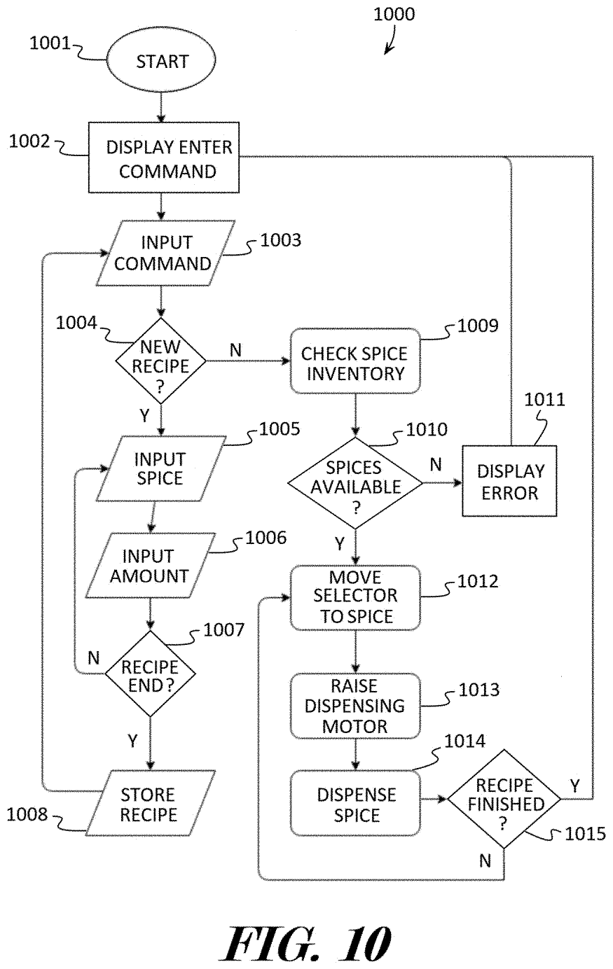

FIG. 10 depicts another example of a method for dispensing edible nutritional items from a dietary metering device (“the method”) 1000 according to various embodiments described herein. The method 1000 may be used by a device 140 as shown in FIG. 9 having a dispensing motor 148A which may be moved to operate the various dispensers 146 of the device 140.

In some embodiments, the method 1000 may start 1001 and an enter command may be displayed to a user in step 1002. For example, an enter command may be displayed on a touch screen display device 152A/input device 152B that instructs the user to select one or more edible nutritional items to be dispensed from the reservoirs 142 of the device 140.

In step 1003, an input command may be provided to the device 140 by the user. For example, the user may operate the touch screen display device 152A/input device 152B to select one or more edible nutritional items to be dispensed from the reservoirs 142 of the device 140.

In decision block 1004, the control unit 150 may determine if the input provided in step 1003 is a new recipe. If the input is a new recipe, the method may proceed to step 1005. If the input is not a new recipe, the method may proceed to step 1009.

In steps 1005 and 1006, the user may use the display device 152A/input device 152B to input a desired edible nutritional item (spice in this example) and a desired amount of the edible nutritional item. In decision block 1007, the method 1000 may continue to step 1005 if there are more edible nutritional items to be entered by the user or to step 1008 if not more edible nutritional items to be entered and the recipe may be stored by the control unit 150. After step 1008, the method 1000 may proceed to step 1003.

In step 1009, the control unit 150 may check the edible nutritional item inventory contained in the reservoirs 142 and proceed to decision block 1010. If edible nutritional item inventory is not sufficient to provide the desired edible nutritional item(s) entered by the user, then the display device 152A may display an error message to the user. If edible nutritional item inventory is sufficient to provide the desired edible nutritional item(s) entered by the user, the method 1000 may proceed to step 1012.

In step 1012, the selector (carousel 161 in this example) may be moved until the dispensing motor 148A is proximate a reservoir 142 having a desired edible nutritional item. In step 1013, the dispensing motor 148A may be raised or otherwise positioned (such as via a small servo or other actuator) in contact with a motivator 147 that is able to move the dispenser 146 of the reservoir 142 having the desired edible nutritional item. In step 1014, the dispensing motor 148A may be operated to cause the dispenser 146 to dispense the desired amount of the edible nutritional item.

Next in decision block 1015, the control unit 150 may determine if the recipe is finished. If the recipe is not finished and requires another edible nutritional item, the method 1000 may continue to step 1012. If the recipe is finished and does not require another edible nutritional item, the method may proceed to step 1002 and the control unit 150 may await further user input by displaying the enter command.

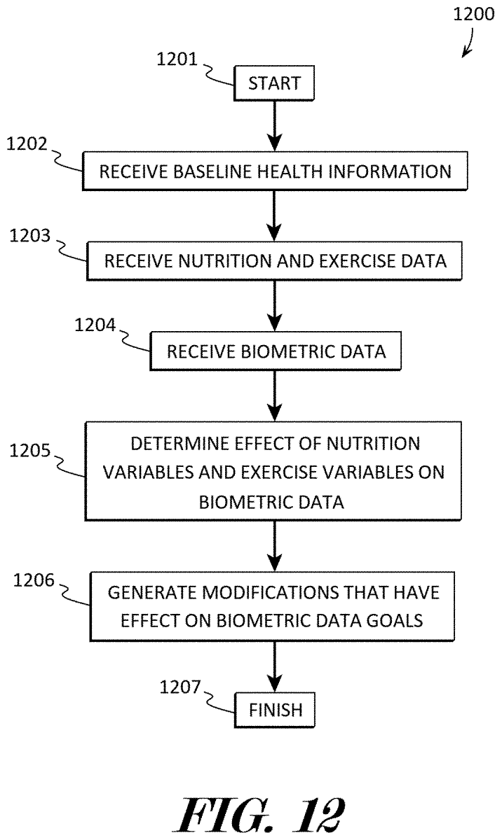

FIG. 12 shows a block diagram of an example of a computer-implemented method for generating modifications (“the method”) 1200 according to various embodiments described herein. In some embodiments, the method 1200 may be used to enable the system 100 to generate one or more modifications for a user 101 using the nutrition data, exercise data, and/or biometric data of the user 101 and/or one or more other users 101. One or more steps of the method 1200 may be performed by one or more health applications 121, acquisition engines 122, and/or an analytics engines 123 which may be executed by a computing device processor, such as a processor 151 (FIG. 6), processor 302 (FIG. 2), and/or a processor 402 (FIG. 3).

The method 1200 may start 1201 and baseline health information of one or more users 101 may be received in step 1202. In some embodiments, the baseline health information may be received from a user 101 via control input(s) 159 by a health application 121 of a device 140. In further embodiments, baseline health information may be received from a user 101 via a health application 121 running on a client device 400 and/or server 300. Generally, the baseline health information may describe the baseline health information of the user 101, such as age, height, weight, glucose levels, blood pressure levels, and cholesterol levels. Preferably, baseline health information may be provided via one or more health monitoring devices 130.

In step 1203, user nutrition and exercise data of one or more users 101 may be received from one or more health monitoring device(s) 130 and/or client devices 400 via user 101 input provided to a health application 121. In some embodiments, the nutrition and exercise data may describe calorie count data and nutritional data of foods eaten by the user 101 over a period of time, such as a breakfast time period, lunch time period, snack time period, dinner time period, and daily time period, which may be entered by the user 101 into a health application 121 running on their client device 400 and/or server 300. In further embodiments, the nutrition and exercise data may comprise data recorded by the one or more health monitoring devices 130 utilized by the user 101. In still further embodiments, the user data may comprise data describing exercise and physical activity of the user 101 over a period of time.

In step 1204, biometric data of one or more users 101 may be received. In some embodiments, an acquisition engine 122 may receive user biometric data from a health monitoring device 130, such as a patch device 130A, blood pressure cuff/machine device 130B, weight scale device 130C, and a standalone device 130D. The biometric data of the user 101 may include a body weight measurement, a cholesterol measurement, a blood pressure measurement, a blood glucose measurement, body fat percentage measurement, or any other human body metric.

In step 1205, the effect of nutrition variables and exercise variables on biometric data of one or more users 101 may be determined by an analytics engine 123. Generally, an analytics engine 123 may use statistical analysis methods to correlate nutrition variables and exercise variables to recorded changes in user biometric data to determine if the nutrition variables and exercise variables increase, decrease, or cause no change to the various user biometric data recorded by the system 100. For example, the analytics engine 123 may determine if an exercise variable of regular jogging causes an increase, decrease, or no change to the blood glucose levels of one or more users 101. As another example, the analytics engine 123 may determine if a nutrition variable of eating 1 gram of fresh ground cinnamon a day, preferably dispensed via a dietary metering device 140, causes an increase, decrease, or no change to the serum cholesterol levels of one or more users 101.

In step 1206, one or more modifications that have an effect on biometric data goals may be generated by an analytics engine 123. In preferred embodiments, one or more modifications provided to a user 101 may be generated using the effect of one or more nutrition variables and/or exercise variables on a set of biometric data recorded from a plurality of users 101. By determining the effect of nutrition variables and exercise variables on biometric data of one or more users 101 in step 1205, the analytics engine 123 may then generate one or more modifications that may have a desired effect on a biometric data goal of a user 101. For example, if the analytics engine 123 determines that an exercise variable of regular jogging causes a decrease to the blood glucose levels of one or more users 101 and a user 101 inputs a biometric data goal of reducing blood glucose levels to a desired number or range, the analytics engine 123 may generate a modification having an exercise recommendation type of health recommendation comprising a jogging regimen to that user 101. As another example, if the analytics engine 123 determines that a nutrition variable of eating 1 gram of fresh ground cinnamon, preferably stored in and dispensed via a dietary metering device 140, causes a decrease to the serum cholesterol levels of one or more users 101 and a user 101 inputs a biometric data goal of reducing serum cholesterol levels to a desired number or range, the analytics engine 123 may generate a modification having a dietary recommendation type of health recommendation comprising dietary regimen of eating 1 gram of fresh ground cinnamon a day to that user 101.

After step 1206, the method 1200 may finish 1207.

It will be appreciated that some exemplary embodiments described herein may include one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the methods and/or systems described herein. Alternatively, some or all functions may be implemented by a state machine that has no stored program instructions, or in one or more application specific integrated circuits (ASICs), in which each function or some combinations of certain of the functions are implemented as custom logic. Of course, a combination of the two approaches may be used. Moreover, some exemplary embodiments may be implemented as a computer-readable storage medium having computer readable code stored thereon for programming a computer, server, appliance, device, etc. each of which may include a processor to perform methods as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), a Flash memory, and the like.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a propagated signal or a computer readable medium. The propagated signal is an artificially generated signal, e.g., a machine generated electrical, optical, or electromagnetic signal that is generated to encode information for transmission to suitable receiver apparatus for execution by a computer. The computer readable medium can be a machine readable storage device, a machine readable storage substrate, a memory device, a composition of matter effecting a machine readable propagated signal, or a combination of one or more of them.

A computer program (also known as a program, software, software application, application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Additionally, the logic flows and structure block diagrams described in this patent document, which describe particular methods and/or corresponding acts in support of steps and corresponding functions in support of disclosed structural means, may also be utilized to implement corresponding software structures and algorithms, and equivalents thereof. The processes and logic flows described in this specification can be performed by one or more programmable processors executing one or more computer programs to perform functions by operating on input data and generating output.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, solid state drives, or optical disks. However, a computer need not have such devices.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube), light emitting diode (LED) display, or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network or the cloud. The relationship of client and server arises by virtue of computer programs running on the respective computers and having a client server relationship to each other.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The computer system may also include a main memory, such as a random access memory (RAM) or other dynamic storage device (e.g., dynamic RAM (DRAM), static RAM (SRAM), and synchronous DRAM (SDRAM)), coupled to the bus for storing information and instructions to be executed by processor. In addition, the main memory may be used for storing temporary variables or other intermediate information during the execution of instructions by the processor. The computer system may further include a read only memory (ROM) or other static storage device (e.g., programmable ROM (PROM), erasable PROM (EPROM), and electrically erasable PROM (EEPROM)) coupled to the bus for storing static information and instructions for the processor.

The computer system may also include a disk controller coupled to the bus to control one or more storage devices for storing information and instructions, such as a magnetic hard disk, and a removable media drive (e.g., floppy disk drive, read-only compact disc drive, read/write compact disc drive, compact disc jukebox, tape drive, and removable magneto-optical drive). The storage devices may be added to the computer system using an appropriate device interface (e.g., small computer system interface (SCSI), integrated device electronics (IDE), enhanced-IDE (E-IDE), direct memory access (DMA), or ultra-DMA).

The computer system may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system may also include a display controller coupled to the bus to control a display, such as a cathode ray tube (CRT), liquid crystal display (LCD), light emitting diode (LED) display, or any other type of display, for displaying information to a computer user. The computer system may also include input devices, such as a keyboard and a pointing device, for interacting with a computer user and providing information to the processor. Additionally, a touch screen could be employed in conjunction with display. The pointing device, for example, may be a mouse, a trackball, or a pointing stick for communicating direction information and command selections to the processor and for controlling cursor movement on the display. In addition, a printer may provide printed listings of data stored and/or generated by the computer system.

The computer system performs a portion or all of the processing steps of the invention in response to the processor executing one or more sequences of one or more instructions contained in a memory, such as the main memory. Such instructions may be read into the main memory from another computer readable medium, such as a hard disk or a removable media drive. One or more processors in a multi-processing arrangement may also be employed to execute the sequences of instructions contained in main memory. In alternative embodiments, hard-wired circuitry may be used in place of or in combination with software instructions. Thus, embodiments are not limited to any specific combination of hardware circuitry and software.

As stated above, the computer system includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of computer readable media, the present invention includes software for controlling the computer system, for driving a device or devices for implementing the invention, and for enabling the computer system to interact with a human user. Such software may include, but is not limited to, device drivers, operating systems, development tools, and applications software. Such computer readable media further includes the computer program product of the present invention for performing all or a portion (if processing is distributed) of the processing performed in implementing the invention.

The computer code or software code of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

Various forms of computer readable media may be involved in carrying out one or more sequences of one or more instructions to processor for execution. For example, the instructions may initially be carried on a magnetic disk of a remote computer. The remote computer can load the instructions for implementing all or a portion of the present invention remotely into a dynamic memory and send the instructions over the air (e.g. through a wireless cellular network or Wi-Fi network). A modem local to the computer system may receive the data over the air and use an infrared transmitter to convert the data to an infrared signal. An infrared detector coupled to the bus can receive the data carried in the infrared signal and place the data on the bus. The bus carries the data to the main memory, from which the processor retrieves and executes the instructions. The instructions received by the main memory may optionally be stored on storage device either before or after execution by processor.

The computer system also includes a communication interface coupled to the bus. The communication interface provides a two-way data communication coupling to a network link that is connected to, for example, a local area network (LAN), or to another communications network such as the Internet. For example, the communication interface may be a network interface card to attach to any packet switched LAN. As another example, the communication interface may be an asymmetrical digital subscriber line (ADSL) card, an integrated services digital network (ISDN) card or a modem to provide a data communication connection to a corresponding type of communications line. Wireless links may also be implemented. In any such implementation, the communication interface sends and receives electrical, electromagnetic or optical signals that carry digital data streams representing various types of information.

The network link typically provides data communication to the cloud through one or more networks to other data devices. For example, the network link may provide a connection to another computer or remotely located presentation device through a local network (e.g., a LAN) or through equipment operated by a service provider, which provides communication services through a communications network. In preferred embodiments, the local network and the communications network preferably use electrical, electromagnetic, or optical signals that carry digital data streams. The signals through the various networks and the signals on the network link and through the communication interface, which carry the digital data to and from the computer system, are exemplary forms of carrier waves transporting the information. The computer system can transmit and receive data, including program code, through the network(s) and, the network link and the communication interface. Moreover, the network link may provide a connection through a LAN to a client device such as a personal digital assistant (PDA), laptop computer, or cellular telephone. The LAN communications network and the other communications networks such as cellular wireless and Wi-Fi networks may use electrical, electromagnetic or optical signals that carry digital data streams. The processor system can transmit notifications and receive data, including program code, through the network(s), the network link and the communication interface.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A computer implemented method for providing dietary and health information, the method comprising the steps of:
receiving input from a user through a client device, wherein the input comprises a biometric data goal;
receiving baseline health information of the user via a health monitoring device, the baseline health information comprising a first biometric data, the first biometric data communicated from the health monitoring device to the client device;
receiving user nutrition and exercise data via the client device; and
providing a first modification to the user via the client device, wherein the first modification comprises a health recommendation to achieve the biometric data goal of the user.

2. The method of claim 1, wherein the health recommendation comprises an exercise recommendation.

3. The method of claim 1, wherein the health recommendation comprises a dietary recommendation.

4. The method of claim 3, wherein the dietary recommendation comprises an amount of an edible nutritional item that is dispensed from a dietary metering device.

5. The method of claim 3, wherein the biometric data goal of the user describes a desired change in the weight of the user, and wherein the dietary recommendation describes a daily number of calories that the user should consume to enable the user to achieve the desired change in the weight of the user.

6. The method of claim 1, wherein the health monitoring device comprises an electronic device selected from the group consisting of a patch device, a blood pressure cuff/machine device, a standalone device, and a weight scale device.

7. The method of claim 1, further comprising the step of recording a second biometric data, the second biometric data taken after a period of time after the first biometric data is received, and further comprising the step of providing a second modification to the user via the client device, the second modification provided after the step of recording the second biometric data.

8. The method of claim 1, wherein the first modification provided to the user is generated using the effect of a variable on a set of biometric data recorded from a plurality of users, the variable selected from the group consisting of a nutrition variable and an exercise variable.

9. The method of claim 1, further comprising the step of providing the health progress of the user via the client device.

10. The method of claim 1, wherein the first biometric data of the user comprises one of a body weight measurement, a cholesterol measurement, a blood pressure measurement, and a blood glucose measurement.

11. A computer implemented system for providing dietary and health information, the system comprising:
a client device having a display screen and an input interface for receiving input from a user; and
a computing platform having a processor, a memory in communication with the processor, and
communication logic stored in the memory, executable by the processor and configured to receive input from the user, wherein the input comprises a biometric data goal, and to receive baseline health information of the user via a health monitoring device, the baseline health information comprising a first biometric data, the first biometric data communicated from the health monitoring device to the client device,
acquisition logic stored in the memory, executable by the processor and configured to receive user nutrition and exercise data via the client device, and
analytics logic stored in the memory, executable by the processor and configured to generate a first modification, wherein the first modification comprises a health recommendation to achieve the biometric data goal of the user, and wherein the first modification is provided to the user via the communication logic.

12. The system of claim 11, wherein the health recommendation comprises an exercise recommendation.

13. The system of claim 11, wherein the health recommendation comprises a dietary recommendation.

14. The system of claim 13, wherein the dietary recommendation comprises an amount of an edible nutritional item that is dispensed from a dietary metering device.

15. The system of claim 13, wherein the biometric data goal of the user describes a desired change in the weight of the user, and wherein the dietary recommendation describes a daily number of calories that the user should consume to enable the user to achieve the desired change in the weight of the user.

16. The system of claim 11, wherein the health monitoring device comprises an electronic device selected from the group consisting of a patch device, a blood pressure cuff/machine device, a standalone device, and a weight scale device.

17. The system of claim 11, further comprising the step of recording a second biometric data, the second biometric data taken after a period of time after the first biometric data is received, and further comprising the step of providing a second modification to the user via the client device, the second modification provided after the step of recording the second biometric data.

18. The system of claim 11, wherein the first modification provided to the user is generated using the effect of a variable on a set of biometric data recorded from a plurality of users, the variable selected from the group consisting of a nutrition variable and an exercise variable.

19. The system of claim 11, further comprising the step of providing the health progress of the user via the client device.

20. The system of claim 11, wherein the first biometric data of the user comprises one of a body weight measurement, a cholesterol measurement, a blood pressure measurement, and a blood glucose measurement.
